(19) United States
(12) Patent Application Publication
(10) Pub. No.: US 2020/0375665 A1
(43) Pub. Date: Dec. 3, 2020

(54) MEDICAL CONTINUUM ROBOT AND METHODS THEREOF

(71) Applicant: Canon U.S.A., Inc., Melville, NY (US)

(72) Inventors: Fumitaro Masaki, Cambridge, MA (US); Brian Ninni, Brighton, MA (US); Takahisa Kato, Brookline, MA (US)

(21) Appl. No.: 16/886,489

(22) Filed: May 28, 2020

Related U.S. Application Data
(60) Provisional application No. 62/855,503, filed on May 31, 2019.

(51) Int. Cl. A61B 34/20; A61B 34/37; A61B 34/00; A61B 1/005; A61B 1/00; A61B 1/267

(52) U.S. Cl. CPC A61B 34/20; A61B 34/37; A61B 34/74; A61B 2034/301; A61B 1/00006; A61B 1/2676; A61B 1/0051

(57) ABSTRACT

The subject disclosure is directed to an articulated medical device in communication with a ventilator, used for regulating breathing patterns in a subject, wherein the articulated medical device may be advanced or retracted during repeatable breathing cycles, so as to reduce the possibility of abrasion to the lungs.

### CROSS REFERENCE TO RELATED PATENT APPLICATIONS

This application claims priority from U.S. Provisional Patent Application No. 62/855,503 filed on May 31, 2019, in the United States Patent and Trademark Office, the disclosure of which is incorporated herein in its entirety by reference.

### FIELD OF DISCLOSURE

The present disclosure relates generally to an articulated SR Robot 20 wherein the device is capable of maneuvering within a subject/patient. The SR Robot 20 may further comprise a hollow cavity, wherein the cavity is configured to allow for medical tool to be guided through the hollow cavity for medical procedures. The tool guided through the cavity may include an endoscope, camera, and/or a catheter, to name a few. More specifically, the subject disclosure details a SR Robot 20 and methods for advancing the SR Robot 20 through the ever-changing anatomy of a subject/patient.

### BACKGROUND OF THE DISCLOSURE

Articulated SR Robots generally include one or more channels that extend along the inside of the device to allow access to end effectors (the actual working part of a surgical instrument or tool) located at a distal end of the SR Robot. Control mechanisms located at a proximal end of the SR Robot are configured to enable remote manipulation of the end effectors via the one or more channels. Accordingly, the mechanical structure of the SR Robot plays a key role in ensuring flexible access to end effectors, while protecting delicate organs and tissues of a patient.

In order to facilitate articulation of these SR Robots, continuum robots are used in clinical cases, especially to articulate around/through organs with tortuous structures, such as the airway of the lung and blood vessel. Clinical studies have shown that robotic bronchoscope can reach higher generation of the airway of the lung than a conventional manual bronchoscope. In addition, a navigation bronchoscopy system, which combines electromagnetic navigation systems with respiratory gating technology, further improve efficacy and use, and allows for the system to display the shape of the airway based on respiratory motion.

In order to control a continuum robot with multiple articulating sections, follow-the-leader (“FTL”) motion is widely used. In FTL, an operator only controls the leading section of the continuum robot, allowing and the rest of articulating sections to automatically follow in the path of the leading section.

By way of example, United Stated Patent Application no. 2012/0059248 to Holsing et al., details a pathway process wherein the respiratory signal is used to gate localization data of the instrument to determine on airway models and correlate the instrument position to the image data to provide a registration of the patient airway models during a respiratory cycle of the patient.

However, the existing art fails to address a relevant and challenging element of the continuum robot process, namely, advancement of the robot in a moving patient. More specifically, as breathing motion, or other voluntary and involuntary motions of a patient, alters the shape of airway during insertion of the continuum robot, the insertion route may be significantly altered with each inhalation and/or exhalation, as well as other patient movements.

Furthermore, breathing motion decreases the advantage of FTL, in that FTL assumes that the shape of surroundings remains constant. However, when following section(s) follow the tip section of the SR Robot, breathing motion changes the shape of the airway. As such, the following section will contact the differing shape of the anatomy.

Accordingly, advancement of the SR Robot may cause abrasion or trauma to the airway if the shape of airway is changed, leading to unnecessary uncomfort in the patient.

### SUMMARY

Thus, to address such exemplary needs in the industry, the present disclosure teaches apparatus, systems and method for a medical apparatus comprising a driving unit; a single sheath that includes at least a first bendable segment which is bendable by the driving unit; a controller configured to send a control signal to the driving unit for bending the first bendable segment; and a device for regulating involuntary motion in a subject; wherein the regulation by the device defines at least two time frames, and wherein the driving unit bends the at least one bendable segment during at least one of the at least two time frames.

In various embodiments, the medical apparatus further comprises a sensor configured for measuring a change in position in the subject. Furthermore, the sensor for measuring the change in position is configured to measure voluntary or involuntary movement.

In yet additional embodiments, the change in position measured in the subject is selected from the group consisting of respiration, digestion, blood circulation, and enzyme production/distribution.

In other embodiments, the device for regulating involuntary motion in a subject is a ventilator.

In additional embodiments, the medical apparatus further comprises a second bendable segment which is bendable by the driving unit. Furthermore, the driving unit can independently control the first bendable segment and second bendable segment.

In additional embodiments, the medical apparatus controller may be configured to control the first bendable segment independent of the second bendable segment, while a force is applied from the controller to the second bendable segment via the driving unit in order to maintain a shape of the second bendable segment.

In additional embodiments, the medical apparatus includes a third bendable segment which is bendable by the driving unit.

In addition, the driving unit can independently control the first bendable segment, second bendable segment and third bendable segment, wherein the first bendable segment and the second bendable segment are configured to independently change a respective bending angle and a respective bending plane in a three dimension space.

In additional embodiments, the medical apparatus controller is configured to dislocate from the sheath at a distal end of the sheath.

In further embodiments, the medical sheath further comprises an outer wall covering at least the first bendable segment. It is further contemplated that the outer wall is configured to attach to the first bendable segment and provide flexible support to the sheath.

In yet additional embodiments, the sheath comprises a hollow cavity extending the length of the sheath for insertion of a medical tool, where in the medical tool is selected from the group consisting of a biopsy tool, an endoscope, a camera, clamps, a grasper, scissors, staplers, variations thereof, and derivatives therefrom.

It is further contemplated that in various embodiment of the subject innovation, the at least two time frames are repeatable in the subject.

The subject innovation further teaches a method for treating a subject, comprising: providing a medical apparatus comprising: a driving unit; a single sheath that includes at least a first bendable segment, which is bendable by the driving unit; a controller configured to send a control signal to the driving unit for bending the first bendable segments; and a device for regulating an involuntary motion in a subject; wherein the regulation by the device defines at least two time frames, and wherein the driving unit bends the at least one bendable segment during at least one of the at least two time frames, affixing the medical device to subject; recording the regulation of movement by the device; identifying the at least two time frames; advancing the first bendable segment in the subject during at least one of the at least two time frames.

In further embodiments, the involuntary motion being regulated in a subject is selected from the group consisting of inhalation, exhalation, and pauses in respiration.

The subject method may also comprise a sensor configured for measuring the involuntary motion in a subject.

In further embodiments of the subject method, advancing the first bendable segment in the subject occurs is selected from the group consisting of maximum inhalation, middle of inhalation and exhalation, inhalation plateau, and exhalation plateau.

These and other objects, features, and advantages of the present disclosure will become apparent upon reading the following detailed description of exemplary embodiments of the present disclosure, when taken in conjunction with the appended drawings, and provided paragraphs.

### BRIEF DESCRIPTION OF THE DRAWINGS

Further objects, features and advantages of the present invention will become apparent from the following detailed description when taken in conjunction with the accompanying figures showing illustrative embodiments of the present invention.

FIG. 1 provides a diagram of the communication of at least two elements of a SR Robot 20, according to one or more embodiments of the present subject matter.

FIG. 2 illustrates a respiratory cycle of a subject/patient, according to one or more embodiments of the present subject matter.

FIG. 3 provides a respiratory cycle of a subject/patient, according to one or more embodiments of the present subject matter.

FIG. 4 provides a respiratory cycle of a subject/patient, according to one or more embodiments of the present subject matter.

Throughout the Figures, the same reference numerals and characters, unless otherwise stated, are used to denote like features, elements, components or portions of the illustrated embodiments. In addition, reference numeral(s) including by the designation “′” (e.g. 12′ or 24′) signify secondary elements and/or references of the same nature and/or kind. Moreover, while the subject disclosure will now be described in detail with reference to the Figures, it is done so in connection with the illustrative embodiments. It is intended that changes and modifications can be made to the described embodiments without departing from the true scope and spirit of the subject disclosure as defined by the appended paragraphs.

### DETAILED DESCRIPTION

The following paragraphs describe certain explanatory embodiments. Other embodiments may include alternatives, equivalents, and modifications. Additionally, the explanatory embodiments may include several novel features, and a particular feature may not be essential to some embodiments of the devices, systems, and methods that are described herein.

The present subject matter discloses apparatus, systems and methods for an articulated SR Robot configured to be guided into a patient or subject (hereafter used interchangeably) and articulated around/through organs and other elements within the patient to reach a desired destination. The SR Robot is designed to navigate through tortuous structures without causing harm or trauma to the patient, as well as being capable of non-invasive advancement in the patient, even if/after the patient has moved.

Movement in a subject, such as breathing motion, decreases the advantage of FTL. FTL assumes that the shape of surroundings remaining constant to when the tip of the FTL advanced through the section. However, when following section(s) follow the tip section of the SR Robot, breathing motion changes the shape of the airway. When the following section(s) are advanced, the following section(s) could result in abrading the airway if the shape of airway is changed, leading to trauma and friction (resistance).

By incorporating a mechanism to monitor and/or record various movements in the patient, the present subject matter can take those recordings into account when advancing the SR Robot through the subject. Although respiratory movement has been focused on in the subject disclosure, any voluntary or involuntary movements in the patient may be accounted for, and adjusted accordingly, when the SR Robot is being advanced. Examples of movement in the patient, which may be accounted for by the subject innovation, include, but are not limited to, respiration, digestion, blood circulation, or enzyme production/distribution.

In order to advance the medical device safely into the patient, a controller 12 records the breathing motion (respiratory phase) of the patient, and synchronizes the breathing motion with advancement/insertion of the medical device 20 (also referred to herein as “SR”, “SR Robot” or “Robot”). For example, the insertion of the SR Robot 20 may be allowed only during a specific time frame/window (e.g. maximum inhalation), as seen in FIG. 2.

In one embodiment, as bronchoscopy is typically done under general anesthesia, a device for regulating involuntary motion in a subject, here a ventilator 14, controls the breathing of the patient. The medical apparatus controller 12 receives breathing signals from the ventilator 14 and inputs the signals to control the SR Robot 20 from a user interface 16. The controller 12 sends a signal to control the SR Robot 20 during a specific time frame, and the input may be ignored and/or delayed when the input is attempted outside the specific time frame.

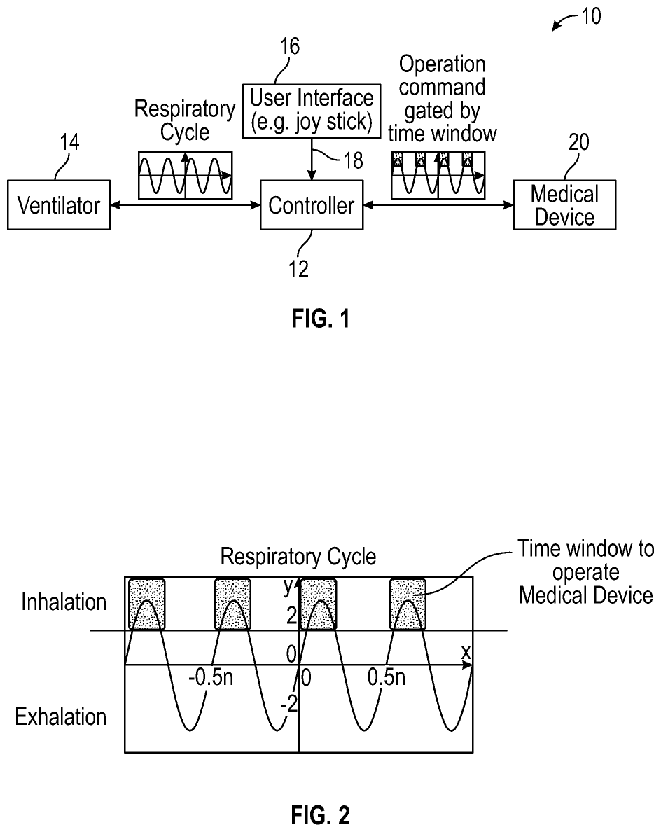

In FIG. 1 we see various elements of the subject medical apparatus 10, according to one or more embodiments of the present subject matter. Accordingly, the user interface 16 is in communication with the controller 12 (“SR controller”) which communicates with the SR Robot 20, which is inserted into the patient. The controller 12 send operational signals from the user interface device 16 to the SR Robot 20, for enacting the SR Robot 20 (insertion/extraction/etc.). The controller 12 is in further communication with a device for regulating involuntary motion in a subject, here a ventilator 14, (“Respiratory Equipment”) and records the respirator cycle of the patient. As discussed previously, the ventilator 14 may be interchanged with any appropriate element depending on the movements in the patient being monitored/recorded and being accounted for; and example may be a heart monitor for cardiac surgery.

In the present example, configured for the lungs and taking into account movement of the patient due to respiration, when the SR Robot 20 is inserted into the patient, a device controller 12 receives operational signals from the user interface device 16 and ventilator 14 (“Respiratory Equipment”), and sends the operation signal to the SR Robot 20. When the controller 12 sends the signal to the SR Robot 20, the controller records the breathing motion (respiratory cycle), and identifies various windows of operation based on the breathing motion, as a history of the insertion.

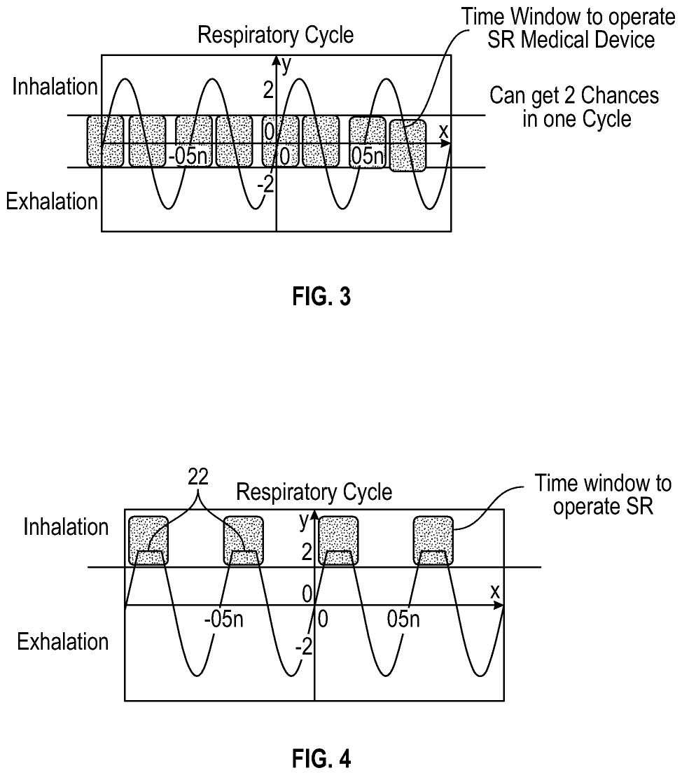

In FIG. 2, the exemplary window of operation (shaded) is identified as the inhalation portion in the breathing cycle of a patient. Alternatively, as seen in FIG. 3, the window of operation can be in the middle of inhalation and exhalation. In the embodiment depicted in FIG. 3, the user has two chances to control the SR 20 compared to the more limited time window provided in the example provided in FIG. 2.

The SR controller 12 may have a monitor (not shown) to aid the end user by showing the breathing cycle. When the breathing cycle arrives at the specific time window, the display may change color to indicate to the user that the SR Robot 20 may be advanced. In this embodiment, the user is enabled to always control SR Robot 20, even if the SR Robot is advanced outside of the time window. In other embodiments, the medical apparatus 10 may be configured to allow the SR Robot to advance only when the time window is applicable.

FIG. 4 depicts yet another embodiment of the subject innovation, wherein the ventilator 14 used in controlling the patient's breathing can create a plateau 22 in the breathing cycle. In this example, the plateau 22 is created around maximal inhalation, and is intended to keep the shape of the airway consistent for safer insertion. The SR controller 12 receives the breathing signals from the ventilator 14, and inputs to control the SR Robot 20 from a user interface device 16. The SR controller 12 then send the signal to control SR Robot 20 during the specific time window. The SR Robot 20 may be configured to ignore the signal 18 when the signal 18 is provided outside the specific time window, thus eliminating the risk of advancing the SR Robot 20 while the anatomy of the patient does not correlate with the intended route.

Alternatively, or supplemental to the above-referenced recorded history used for advancing the SR Robot 20, variables in force measured against time, caused by interactions between the SR Robot 20 and the airway, may be used to determine the appropriate advancement process. For example, when the SR Robot 20 remains at a certain point within the patient, a force caused by interaction between the SR Robot 20 and the airway may be recorded and measured. The force may be periodic, which correlates with breathing motion, as the breathing motion deforms the airway, and the deformation compresses the SR Robot 20. Sensors 24 (not shown) within the SR Robot 20 can be utilized to measure the force and determine variations in time, which would correlate to inflation and deflation of the lungs, signifying breathing.

In addition, a force sensor 24 (not shown) may be attached to the SR Robot 20. Accordingly, as the SR Robot 20 is being advanced, the controller 12 receives a signal from the user interface 16, and a force is recorded via the data acquisition system (“DAQ”), which sends the operation signal 18 to the SR Robot 20. When the controller 12 sends a signal 18 to SR Robot 20, the controller 12 records the force, as well as the position of the sensor 24.

In further embodiments, the controller 12 synchronizes the breathing motion with insertion of the SR Robot 20. The insertion of the SR Robot 20 is allowed only during a designated time window (e.g. maximum inhalation).

As before, the controller 12 may incorporate or communicate with a monitor (not shown) that shows the breathing cycle. When the breathing cycle comes to the specific time window of the breathing cycle, the display may change color, or provide other visual indicator(s), to convey to the user that the user may manipulate the SR Robot 20. In various embodiment, the user may always be able to manipulate the SR Robot, even outside of the time window, and in others, manipulation may be restricted.

In a further embodiment, the controller 12 may synchronizes the breathing motion with insertion of the SR Robot 20. The insertion of SR Robot 20 may be arranged for insertion at a specific time window (e.g. maximum inhalation). As stated earlier, bronchoscopy is typically done under general anesthesia, so a ventilator 14 controls the breathing of the patient. The ventilator 14 creates the plateau 22 in the breathing cycle around the maximal inhalation, and keeps the shape of the airway consistent. The controller 12 receives signals of breathing from the ventilator 14 and input to control SR Robot 20 from a user interface 16. The SR controller 12 send a signal 18 to the SR Robot 20 during a specific time window. The SR Robot 20 may be configured to ignore the signal 18 when the signal 18 is provided outside a specific time window. Alternatively, the pause/plateau in ventilation may be controlled during a different time window, for example, when the clinician resumes inserting the SR Robot 20, the ventilator 14 could pause for a certain amount of time. Presumably, the clinician will decide to begin insertion when the airways are in a desirable position, so it would make sense to pause the respiration at this step. The pause should last for as long as the insertion motion is happening (or as long as medically acceptable).

1. A medical apparatus comprising:
a driving unit;
a single sheath that includes at least a first bendable segment which is bendable by the driving unit;
a controller configured to send a control signal to the driving unit for bending the first bendable segment; and
a device for regulating involuntary motion in a subject;
wherein the regulation by the device defines at least two time frames, and
wherein the driving unit bends the at least one bendable segment during at least one of the at least two time frames.

2. The medical apparatus of claim 1, further comprising a sensor configured for measuring a change in position in the subject.

3. The medical apparatus of claim 2, wherein the sensor for measuring the change in position is configured to measure voluntary or involuntary movement.

4. The medical apparatus of claim 2, wherein the change in position measured in the subject is selected from the group consisting of respiration, digestion, blood circulation, and enzyme production/distribution.

5. The medical apparatus of claim 1, wherein the device is a ventilator

6. The medical apparatus of claim 1, further comprising a second bendable segment which is bendable by the driving unit.

7. The medical apparatus of claim 6, wherein the driving unit can independently control the first bendable segment and second bendable segment.

8. The medical apparatus of claim 6, wherein the controller is configured to control the first bendable segment independent of the second bendable segment, while a force is applied from the controller to the second bendable segment via the driving unit in order to maintain a shape of the second bendable segment.

9. The medical apparatus of claim 6, further comprising a third bendable segment which is bendable by the driving unit.

10. The medical apparatus of claim 9, wherein the driving unit can independently control the first bendable segment, second bendable segment and third bendable segment.

11. The medical apparatus of claim 6, wherein the first bendable segment and the second bendable segment are configured to independently change a respective bending angle and a respective bending plane in a three dimension space.

12. The medical apparatus of claim 1, wherein the controller is configured to dislocate from the sheath at a distal end of the sheath.

13. The medical apparatus of claim 1, wherein the sheath further comprises an outer wall covering at least the first bendable segment.

14. The medical apparatus of claim 13, wherein the outer wall is configured to attach to the first bendable segment and provide flexible support to the sheath.

15. The medical apparatus for claim 1, wherein the sheath comprises a hollow cavity extending the length of the sheath for insertion of a medical tool.

16. The medical apparatus of claim 15, where in the medical tool is selected from the group consisting of a biopsy tool, an endoscope, a camera, clamps, a grasper, scissors, staplers, variations thereof, and derivatives therefrom.

17. The medical apparatus for claim 1, wherein the at least two time frames are repeatable in the subject.

18. A method for treating a subject, comprising:
providing a medical apparatus comprising:
a driving unit;
a single sheath that includes at least a first bendable segment,
which is bendable by the driving unit;
a controller configured to send a control signal to the driving unit for bending the first bendable segments; and
a device for regulating an involuntary motion in a subject;
wherein the regulation by the device defines at least two time frames, and
wherein the driving unit bends the at least one bendable segment during at least one of the at least two time frames,
affixing the medical device to subject;
recording the regulation of movement by the device;
identifying the at least two time frames;
advancing the first bendable segment in the subject during at least one of the at least two time frames.

19. The method of claim 18, wherein the involuntary motion being regulated in a subject is selected from the group consisting of inhalation, exhalation, and pauses in respiration.

20. The method of claim 18, further comprising a sensor configured for measuring the involuntary motion in a subject.

21. The method of claim 18, wherein advancing the first bendable segment in the subject occurs is selected from the group consisting of maximum inhalation, middle of inhalation and exhalation, inhalation plateau, and exhalation plateau.
